(19) United States
(12) Patent Application Publication
(10) Pub. No.: US 2007/0292381 A1
(43) Pub. Date: Dec. 20, 2007

(54) Composition and process for coating keratin fibers

(76) Inventor: Isabelle Jacquier, Chevilly Larue (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/312,338

(22) Filed: Dec. 21, 2005

Related U.S. Application Data
(60) Provisional application No. 60/639,796, filed on Dec. 29, 2004.

(30) Foreign Application Priority Data
Dec. 21, 2004 (FR) 04 53117

(51) Int. Cl. A61K 8/88; A61K 8/72; A61K 8/81

(52) U.S. Cl. 424/70.15; 424/70.11; 424/70.16; 424/70.17

(57) ABSTRACT

The present disclosure relates to a composition for coating keratin fibers, comprising, in a cosmetically acceptable medium: a non-aqueous solvent phase, a first polymer comprising a) a polymer skeleton, having hydrocarbon-based repeating units comprising at least one hetero atom, and optionally b) at least one pendent fatty chain and/or at least one terminal fatty chain, which are optionally functionalized, comprising at least 4 carbon atoms and linked to the hydrocarbon-based units, the composition being wax-free.

This application claims benefit of U.S. Provisional Application No. 60/639,796, filed Dec. 29, 2004, the contents of which are incorporated herein by reference. This application also claims benefit of priority under 35 U.S.C. § 119 to French Patent Application No. 04 53117, filed Dec. 21, 2004, the contents of which are also incorporated herein by reference.

The present disclosure relates to a composition for coating keratin fibers, for instance the eyelashes, the eyebrows and the hair. At least one embodiment of the present disclosure relates to a composition for coating the eyelashes.

The composition according to the present disclosure may be in the form of a mascara, a product for the eyebrows, an eyeliner or a hair makeup product. In at least one embodiment, the composition is in the form of a mascara. The composition may be a makeup composition, a composition to be applied over or under a makeup, also known, respectively, as a “top coat” or a “base coat”, or alternatively a composition for treating the eyelashes.

In general, compositions for making up keratin fibers, such as the eyelashes, comprise at least one wax or a mixture of waxes dispersed in a liquid solvent phase.

Anhydrous mascaras or mascaras with a low content of water and/or of water-soluble solvents, which are known as “waterproof mascaras”, formulated in the form of a dispersion of waxes in non-aqueous solvents, and which show good resistance to water and/or to sebum, are also known.

It is mainly by means of the amount of wax, which makes it possible to structure the composition, that the application specificities desired for the compositions are adjusted, for instance their fluidity or consistency, their covering power and/or their curling power, and also their thickening power (also known as charging power or makeup power).

An observation by microscope shows that, in this type of composition, the wax particles are generally in the form of aggregates, which make the composition opaque and reduce its gloss.

It is thus desirable to obtain compositions with good staying power and/or good resistance to water, sweat and sebum, while at the same time being glossy and/or allowing the deposition of a smooth and uniform film onto keratin fibers.

The inventor has discovered, surprisingly, that at least one of these advantages can be achieved by a composition comprising a non-aqueous solvent phase and a structuring polymer.

For example, the composition may be easy to apply and form on the keratin fibers a smooth, uniform, glossy and non-opaque film. The film of the composition may show good staying power over time and good resistance to water and/or sebum.

Furthermore, the makeup may be sparingly charging, i.e., it may not thicken the eyelashes, thus, providing a natural makeup effect.

Accordingly, the present disclosure relates to a composition for coating keratin fibers, comprising, in a cosmetically acceptable medium:

a non-aqueous solvent phase,

at least one polymer comprising a) a polymer skeleton having hydrocarbon-based repeating units comprising at least one heteroatom, and optionally b) at least one pendent fatty chain and/or at least one terminal fatty chain, which are optionally functionalized, comprising at least 4 carbon atoms and being linked to the hydrocarbon-based units,

the composition being wax-free and comprising less than 20% of water and/or of water-soluble solvent.

The present disclosure also relates to a composition for coating keratin fibers, comprising, in a cosmetically acceptable medium:

a non-aqueous solvent phase,

at least one polymer comprising a) a polymer skeleton having hydrocarbon-based repeating units comprising at least one heteroatom, and optionally b) at least one pendent fatty chain and/or at least one terminal fatty chain, which are optionally functionalized, comprising at least 4 carbon atoms and being linked to the hydrocarbon-based units,

the composition having a solids content of less than or equal to 37% by weight relative to the total weight of the composition.

The present disclosure further relates to a composition for coating keratin fibers, comprising, in a cosmetically acceptable medium:

a non-aqueous solvent phase,

at least one first polymer comprising a) a polymer skeleton having hydrocarbon-based repeating units comprising at least one heteroatom, and optionally b) at least one pendent fatty chain and/or at least one terminal fatty chain, which are optionally functionalized, comprising at least 4 carbon atoms and being linked to these hydrocarbon-based units,

at least one second polymer chosen from liposoluble or lipophilic film-forming polymers present in a solids content of greater than or equal to 10% by weight relative to the total weight of the composition.

The present disclosure additionally relates to a cosmetic process for treating or making up keratin fibers, comprising the application of a composition as defined above to the keratin fibers. The present disclosure also relates to the use of a composition as defined above, for obtaining, after depositing on keratin fibers, a film of makeup that has good staying power and/or that is resistant to water and/or sebum.

In at least one embodiment, the composition according to the present disclosure may be a colored or uncolored top coat, to be applied over a first coat of a first composition and intended to increase the staying power and/or the resistance to water and/or sebum of the said first composition. The first composition may be any commercially available mascara usually used by consumers: the composition according to the present disclosure thus makes it possible to improve the staying power of the film of first composition onto which it is applied.

For example, when the composition according to the present disclosure is uncolored, it allows, by virtue of its transparency, the first film of a first composition to be distinguished.

Accordingly, the present disclosure also relates to a process for coating keratin fibers, such as the eyelashes, which comprises applying to the keratin fibers:

i) at least one first coat of a first composition,

ii) and then, after partial or total drying of the first coat, at least one second coat of a second composition comprising
a non-aqueous solvent phase,
at least one polymer comprising a) a polymer skeleton having hydrocarbon-based repeating units comprising at least one heteroatom, and optionally b) at least one pendent fatty chain and/or at least one terminal fatty chain, which are optionally functionalized, comprising at least 4 carbon atoms and being linked to the hydrocarbon-based units,
the second composition being wax-free.

According to one embodiment, the composition according to the present disclosure may be applied over a first coat of a composition, such as, for example, an aqueous mascara, known as “cream mascara”, comprising a high content of water and/or water-soluble solvent, generally greater than or equal to 30% by weight, such as, for example, greater than or equal to 40% by weight or greater than or equal to 50% by weight relative to the total weight of the composition, in order to render the mascara film impermeable.

Accordingly, the present disclosure further relates to a process for coating keratin fibers, such as the eyelashes, which comprises applying to the keratin fibers:

i) at least one first coat of a first composition comprising at least 30% by weight of water and/or of water-soluble solvent,

ii) and then, after partial or total drying of the first coat, at least one second coat of a second composition comprising
a non-aqueous solvent phase,
at least one polymer comprising a) a polymer skeleton having hydrocarbon-based repeating units comprising at least one heteroatom, and optionally b) at least one pendent fatty chain and/or at least one terminal fatty chain, which are optionally functionalized, comprising at least 4 carbon atoms and being linked to the hydrocarbon-based units.

The present disclosure also relates to a makeup kit comprising, in separate packages or compartments:

a first composition comprising at least 30% of water and/or of water-soluble solvent, and

a second composition comprising
a non-aqueous solvent phase,
at least one polymer comprising a) a polymer skeleton having hydrocarbon-based repeating units comprising at least one heteroatom, and optionally b) at least one pendent fatty chain and/or at least one terminal fatty chain, which are optionally functionalized, comprising at least 4 carbon atoms and being linked to the hydrocarbon-based units.

According to another embodiment of the present disclosure, the composition according to the present disclosure may be suitable for application over a first coat of a first composition having a relatively low solids content (or dry extract), for example less than or equal to 50%, which allows the production of a natural, sparingly charging film of makeup, in order to render the first coat of the first composition impermeable.

Accordingly, the present disclosure also relates to a process for coating keratin fibers, such as the eyelashes, which comprises applying to the keratin fibers:

i) at least one first coat of a first composition with a dry extract of less than or equal to 50%, such as less than or equal to 47% or less than or equal to 45%,

ii) and then, after partial or total drying of the first coat, at least one second coat of a second composition comprising
a non-aqueous solvent phase,
at least one polymer comprising a) a polymer skeleton having hydrocarbon-based repeating units comprising at least one heteroatom, and optionally b) at least one pendent fatty chain and/or at least one terminal fatty chain, which are optionally functionalized, comprising at least 4 carbon atoms and being linked to the hydrocarbon-based units.

The present disclosure also relates to a makeup kit comprising, in separate packages or compartments:

a first composition with a dry extract of less than or equal to 50%, such as, for example, less than or equal to 47% or less than or equal to 45%,

a second composition comprising
a non-aqueous solvent phase,
at least one polymer comprising a) a polymer skeleton having hydrocarbon-based repeating units comprising at least one heteroatom, and optionally b) at least one pendent fatty chain and/or at least one terminal fatty chain, which are optionally functionalized, comprising at least 4 carbon atoms and being linked to these hydrocarbon-based units.
   Characterization of the Solids Content

For the purposes of the present disclosure, the term “solids content” denotes the amount of non-volatile material present in the composition.

This amount of solids in the compositions, commonly known as the “dry extract” or its abbreviated form DE, is measured by heating the sample with infrared rays with a wavelength of 2 μm to 3.5 μm. The substances contained in the compositions that have a high vapor pressure evaporate under the effect of this radiation. Measurement of the weight loss of the sample makes it possible to determine the “dry extract” of the composition. These measurements are performed using an LP16® commercial infrared desiccator from Mettler. This technique is fully described in the machine documentation supplied by Mettler.

The measuring protocol is as follows:

About 1 g of the composition is spread onto a metal crucible. After introducing this crucible into the desiccator, it is subjected to a set temperature of 120° C. for one hour. The wet mass of the sample, corresponding to the initial mass, and the dry mass of the sample, corresponding to the mass after exposure to the radiation, are measured using a precision balance.

The solids content is calculated in the following manner:
Dry extract=100×(dry mass/wet mass).

The values measured by means of the protocol described above may differ from the corresponding theoretical values by plus or minus 1%.

In at least one embodiment, the compositions according to the present disclosure may have a solids content of less than or equal to 37% by weight such as, for example, less than or equal to 35% by weight or less than or equal to 33% by weight relative to the total weight of the composition.

The at Least One (First) Polymer

The at least one polymer, also called “the at least one first polymer” in embodiments having at least one second polymer, of the composition according to the present disclosure may have a structuring role and serve to gel the non-aqueous solvent phase (or oily phase) of the composition. For the purposes of the present disclosure, the term “gelled non-aqueous solvent phase” means a non-aqueous solvent phase whose viscosity is increased by adding the at least one (first) polymer, and which flows under its own weight.

The at least one (first) polymer may be, in at least one embodiment, solid and undeformable at room temperature (25° C.) and may be capable of structuring the composition without opacifying it.

In at least one embodiment of the present disclosure, the at least one (first) polymer comprises a) a polymer skeleton having hydrocarbon-based repeating units comprising at least one heteroatom, and optionally b) at least one pendent fatty chain and/or at least one terminal fatty chain, which are optionally functionalized, comprising at least 4 carbon atoms, and being linked to the hydrocarbon-based units via at least one bonding group.

As used herein, the expression “functionalized chain” means an alkyl chain comprising one or more functional or reactive groups chosen from, for example, amide, hydroxyl, ether, oxyalkylene, polyoxyalkylene and halogen groups, including fluoro or perfluoro groups, ester, siloxane and polysiloxane groups. In addition, the hydrogen atoms of one or more fatty chains may be substituted at least partially with fluorine atoms.

For the purposes of the present disclosure, the term “polymer” means a compound comprising at least 2 repeating units, such as, for example, at least 3 repeating units.

In the present disclosure, the expression “hydrocarbon-based repeating units” means a unit comprising from 2 to 80 carbon atoms, for example, from 2 to 60 carbon atoms, bearing hydrogen atoms and optionally oxygen atoms, which may be linear, branched or cyclic, and saturated or unsaturated. These units each also comprise at least one heteroatom that may be non-pendent but is in the polymer skeleton. These heteroatoms may be chosen from nitrogen, sulfur and phosphorus atoms and combinations thereof, optionally combined with one or more oxygen atoms. For example, the units may comprise at least one nitrogen atom, such as a non-pendent nitrogen atom. These units may also comprise a carbonyl group.

The units comprising a heteroatom may be amide units forming a skeleton of the polyamide type, carbamate and/or urea units forming a polyurethane, polyurea and/or polyurea-urethane skeleton. In at least one embodiment of the present disclosure, these units are amide units. The pendent chains may be linked directly to at least one heteroatom of the polymer skeleton.

Between the hydrocarbon-based units, the at least one (first) polymer may comprise silicone units or oxyalkylene units.

In addition, the at least one (first) polymer in the composition of the present disclosure may comprise from 40% to 98%, such as, for example, from 50% to 95%, units comprising fatty chains relative to the total number of units comprising a heteroatom and fatty chains. The nature and proportion of the units comprising a heteroatom may depend on the nature of the fatty phase and may be similar to the polar nature of the fatty phase. Thus, the more the units comprising a heteroatom are polar and in high proportion in the at least one (first) polymer, which corresponds to the presence of several heteroatoms, the greater the affinity may be of the at least one (first) polymer for polar oils. On the other hand, the less polar or even apolar the units comprising a heteroatom or the lower their proportion, the greater the affinity may be of the at least one (first) polymer for apolar oils.

The pendent fatty chains may be linked to at least one of the nitrogen atoms of the amide units of the at least one (first) polymer.

For example, the fatty chains of this polyamide may be present in an amount ranging from 40% to 98%, such as, for example, from 50% to 95%, of the total number of amide units and of fatty chains.

In at least one embodiment, the at least one (first) polymer, such as, for example, where the at least one (first) polymer is a polyamide, of the composition according to the present disclosure may have a weight-average molecular mass of less than 100,000 (e.g., ranging from 1000 to 100,000), such as, for example, less than 50,000 (e.g., ranging from 1000 to 50,000) or ranging from 1000 to 30,000, from 2000 to 20,000 or from 2000 to 10,000.

In at least one embodiment, the at least one (first) polymer, such as, for example, when the at least one (first) polymer is a polyamide, may be insoluble in water, such, as for example, at 25° C. In further embodiments, the at least one (first) polymer does not comprise any ionic groups.

Among the (first) polymers that may be used in the composition, non-limiting mention may be made of polyamides branched with pendent fatty chains and/or terminal fatty chains comprising from 6 to 120 carbon atoms, such as from 8 to 120 or from 12 to 68 carbon atoms, each terminal fatty chain being linked to the polyamide skeleton via at least one bonding group. The bonding group may be chosen from ester, ether, amine, urea, urethane, thioester, thioether, thiourea and thiourethane groups. These polymers may comprise a fatty chain at each end of the polymer skeleton, such as, for example, the polyamide skeleton.

The (first) polymers may be polymers resulting from a polycondensation between a dicarboxylic acid comprising at least 32 carbon atoms (comprising, e.g., from 32 to 44 carbon atoms) and an amine chosen from diamines comprising at least 2 carbon atoms (e.g., from 2 to 36 carbon atoms) and triamines comprising at least 2 carbon atoms (e.g., from 2 to 36 carbon atoms). The diacid may be, for example, a dimer derived from an ethylenically unsaturated fatty acid comprising at least 16 carbon atoms such as from 16 to 24 carbon atoms, for instance oleic acid, linoleic acid or linolenic acid. The diamine may be, for example, ethylenediamine, hexylenediamine or hexamethylenediamine. The triamine may be ethylenetriamine, for example. Polymers comprising one or 2 terminal carboxylic acid groups may be esterified with a monoalcohol comprising at least 4 carbon atoms, such as from 10 to 36 carbon atoms, for example, from 12 to 24 or from 16 to 24 carbon atoms, including, for example 18 carbon atoms.

The polyamide in the composition according to the present disclosure may be chosen from polymers of formula (II) below: 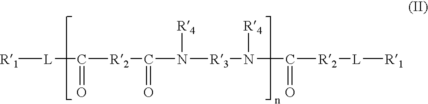
wherein:

n is an integer ranging from 1 to 30,

R′₁, which may be the same or different, are independently chosen from fatty chains chosen from alkyl and alkenyl groups comprising at least 1 carbon atom, such as, for example, from 4 to 24 carbon atoms;

R′₂, which may be the same or different, are independently chosen from hydrocarbon-based radicals comprising from 1 to 52 carbon atoms;

R′₃, which may be the same or different, are independently chosen from organic groups comprising at least one atom chosen from carbon, hydrogen and nitrogen atoms, with the proviso that R′₃ comprises at least 3 carbon atoms;

R′₄, which may be the same or different, are independently chosen from a hydrogen atom, alkyl groups comprising from 1 to 10 carbon atoms, and a direct bond to at least one group chosen from R′₃ and another R′₄ such that the nitrogen atom to which both R′₃ and R′₄ are attached forms part of a heterocyclic structure defined by R′₄—N—R′₃, with the proviso that at least 50% of the R′₄ are hydrogen atoms; and

L is a bonding group chosen from ester, ether, amine, urea, urethane, thioester, thioether, thiourea and thiourethane groups, optionally substituted with at least one group R′₁.

According to one embodiment, these polymers may be chosen from the polymers of formula (I) in which the bonding group L is an ester group

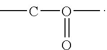

In at least one embodiment of the present disclosure, these polymers may be chosen from those disclosed in U.S. Pat. No. 5,783,657 from the company Union Camp. Each of these polymers is a polymer of formula (I) below: 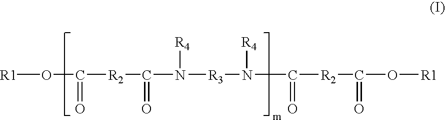
wherein:

m is a whole number of amide units such that the number of ester groups represents from 10% to 50% of the total number of ester and amide groups;

R₁, which may be the same or different, are independently chosen from an alkyl and alkenyl groups comprising at least 4 carbon atoms, such as, for example, from 4 to 24 carbon atoms;

R₂, which may be the same or different, are independently chosen from C₄ to C₄₂ hydrocarbon-based groups, with the proviso that 50% of the groups R₂ are chosen from C₃₀ to C₄₂ hydrocarbon-based groups;

R₃, which may be the same or different, are independently chosen from organic groups comprising at least 2 carbon atoms, hydrogen atoms and optionally at least one oxygen or nitrogen atom; and

R₄, which may be the same or different, are independently chosen from a hydrogen atom, C₁ to C₁₀ alkyl groups and a direct bond to an R₃ group or to another R₄ group, such that the nitrogen atom to which both R₃ and R₄ are attached forms part of a heterocyclic structure defined by R₄—N—R₃, with the proviso that at least 50% of the groups R₄ are hydrogen atoms.

In embodiments where the at least one (first) polymer is chosen from polymers of formula (I), the optionally functionalized terminal fatty chains may be terminal chains linked to the last heteroatom, i.e., nitrogen, of the polyamide skeleton.

The ester groups of formula (I), which form part of the terminal and/or pendent fatty chains, may represent from 15% to 40%, such as, for example, from 20% to 35%, of the total number of ester and amide groups.

In a further embodiment, m may be chosen from an integer ranging from 1 to 5, such as, for example, from 2 to 5.

In at least one embodiment, R₁ is chosen from C₁₂ to C₂₂, such as C₁₆ to C₂₂, alkyl groups. In these embodiments, R₂ can be chosen from C₁₀ to C₄₂ hydrocarbon-based (e.g., alkylene) groups. In further embodiments, at least 50%, such as at least 75%, of the groups R₂ are chosen from groups comprising from 30 to 42 carbon atoms. The other groups R₂ in the further embodiments may be chosen from C₄ to C₁₉, such as C₄ to C₁₂, hydrogen-comprising groups.

In some embodiments, R₃ is chosen from C₂ to C₃₆ hydrocarbon-based groups and polyoxyalkylene groups, and R₄ is a hydrogen atom. In further embodiments, R₃ may be chosen from C₂ to C₁₂ hydrocarbon-based groups.

The hydrocarbon-based groups may be linear, cyclic or branched, and saturated or unsaturated groups. Alkyl and alkylene groups may be linear or branched, and saturated or unsaturated groups.

The polymers of formula (I) may be in the form of mixtures of polymers, these mixtures also possibly comprising a synthetic product corresponding to a compound of formula (I) in which n is 0, i.e., a diester.

As examples of the (first) polymers of formula (I) that may be used in the composition according to the present disclosure, non-limiting mention may be made of the commercial products sold by the company Arizona Chemical under the name UNICLEAR, such as UNICLEAR 80 and UNICLEAR 80V, and UNICLEAR 100, UNICLEAR 100V and UNICLEAR 100VG, which are sold, in the form of an 80% (in terms of active material) gel in a mineral oil and a 100% (in terms of active material) gel. They have a softening point of from 88° C. to 94° C. These commercial products are a mixture of copolymers of a C₃₆ diacid condensed with ethylenediamine, having a weight-average molecular mass of about 6000. The terminal ester groups result from the esterification of the remaining acid end groups with cetyl alcohol, stearyl alcohol or mixtures thereof (also known as cetylstearyl alcohol).

As (first) polymers corresponding to the general formula (II), non-limiting mention may also be made of polymers comprising at least one terminal fatty chain linked to the polymer skeleton via at least one tertiary amide bonding group (also known as amide-terminated polyamide or ATPA).

Such polymers are available, for example, under the name SYLVACLEAR A 200 V from the company Arizona Chemical; see also U.S. Pat. No. 6,503,522.

According to one embodiment, the (first) polymer of formula (II) may also comprise at least one terminal fatty chain linked to the polymer skeleton via at least one ether or polyether bonding group (it is then known as an ether-terminated poly(ether)amide). Such polymers are described, for example, in U.S. Pat. No. 6,399,713.

As (first) polymers that may be used according to the present disclosure, non-limiting mention may also be made of polyamide resins resulting from the condensation of an aliphatic dicarboxylic acid and a diamine (including compounds comprising more than 2 carbonyl groups and 2 amine groups), the carbonyl and amine groups of adjacent individual units being condensed via an amide bond. These polyamide resins may include those sold under the brand name VERSAMID by the companies General Mills Inc. and Henkel Corp. (VERSAMID 930, 744 or 1655) or by the company Olin Mathieson Chemical Corp. under the brand name Onamid®, such as Onamid® S or C. These resins have a weight-average molecular mass ranging from 6000 to 9000. These polyamides are disclosed in U.S. Pat. Nos. 3,645,705 and 3,148,125. In at least one embodiment of the present disclosure, Versamid® 930 or 744 can be used.

It is also possible to use as (first) polymer a poly(esteramide) comprising ester end groups (ester-terminated poly(esteramide) or ETPEA), for instance those whose preparation is described in U.S. Pat. No. 6,552,160. An example of this polyamide is SYLVACLEAR C 75 V from Arizona Chemical.

The polyamides sold by the company Arizona Chemical under the names UNI-REZ (2658, 2931, 2970, 2621, 2613, 2624, 2665, 1554, 2623 and 2662) and the product sold under the name MACROMELT 6212 by the company Henkel may also be used. These polyamides are disclosed in U.S. Pat. No. 5,500,209.

It is also possible to use polyamide resins obtained from plants, such as those disclosed in U.S. Pat. Nos. 5,783,657 and 5,998,570.

The at least one (first) polymer may have a softening point of greater than 65° C., which may be up to 190° C. For example, the at least one (first) polymer may have a softening point ranging from 70° C. to 130° C. such as from 80° C. to 105° C. The at least one (first) polymer may be a non-waxy polymer.

In at least one embodiment, the at least one (first) polymer may be a polymer of formula (I) mentioned above. On account of its fatty chain(s), this (first) polymer is readily soluble in oils and may lead to compositions that are macroscopically homogeneous even with a high content (e.g., at least 25%) of polymer, unlike polymers not comprising a fatty chain.

The at least one (first) polymer may be present in the composition in an amount ranging from 0.01% to 20% by weight relative to the total weight of the composition, such as from 0.05% to 15% by weight or from 0.1% to 10% by weight.

Non-Aqueous Solvent Phase

The composition according to the present disclosure also comprises a non-aqueous solvent phase.

The non-aqueous solvent phase may be capable of forming a continuous phase and contains, as its name indicates, at least one non-aqueous solvent that may be a volatile compound, which is insoluble in water and liquid at room temperature and atmospheric pressure.

For the purposes of the present disclosure, the term “volatile compound” means any compound (or non-aqueous medium) capable of evaporating on contact with the keratin fibers in less than one hour, at room temperature and atmospheric pressure. The volatile compound is a volatile cosmetic compound, which is liquid at room temperature, having a non-zero vapor pressure, at room temperature and atmospheric pressure, such as a vapor pressure ranging from 0.13 Pa to 40 000 Pa (10⁻³ to 300 mmHg). For example, the volatile compound may have a vapor pressure ranging from 1.3 Pa to 13 000 Pa (0.01 to 100 mmHg) or ranging from 1.3 Pa to 1300 Pa (0.01 to 10 mmHg).

In contrast, the term “non-volatile compound” means a compound that remains on the keratin fibers at room temperature and atmospheric pressure at least for several hours and that may have a vapor pressure of less than 10⁻³ mmHg (0.13 Pa).

In at least one embodiment, the amount of water-insoluble volatile compound that is liquid at room temperature may range from 5% to 90%, such as from 10% to 80% or from 20% to 75% by weight relative to the total weight of the composition.

The water-insoluble volatile compound that is liquid at room temperature may be a cosmetically acceptable organic solvent or oil (fatty substance that is liquid at 25° C. and atmospheric pressure). The term “cosmetically acceptable” means a compound whose use is compatible with application to keratin fibers.

Needless to say, the non-aqueous solvent phase of the composition according to the present disclosure may comprise a mixture of such compounds.

The volatile oils may be hydrocarbon-based oils, silicone oils, fluoro oils or mixtures thereof.

The term “hydrocarbon-based oil” means an oil mainly comprising hydrogen and carbon atoms and possibly oxygen, nitrogen, sulfur or phosphorus atoms. The volatile hydrocarbon-based oils may be chosen from hydrocarbon-based oils comprising from 8 to 16 carbon atoms, such as C₈-C₁₆ branched alkanes, for instance C₈-C₁₆ isoalkanes of petroleum origin (also known as isoparaffins), for instance isododecane (also known as 2,2,4,4,6-pentamethylheptane), isodecane, isohexadecane and, for example, the oils sold under the trade names “Isopar®” or “Permethyl®”, branched C₈-C₁₆ esters and isohexyl neopentanoate, and mixtures thereof. Other volatile hydrocarbon-based oils, for instance petroleum distillates, such as those sold under the name “Shell Solt®” by the company Shell, may also be used.

Volatile silicones may also be used as volatile oils, for instance volatile linear or cyclic silicone oils, including those with a viscosity ≦6 centistokes (6×10⁻⁶ m²/s) and, for example, comprising from 2 to 10 silicon atoms, these silicones optionally comprising alkyl or alkoxy groups comprising from 1 to 22 carbon atoms. As volatile silicone oils that may be used in the present disclosure, non-limiting mention may be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile organic solvents, including fluorinated volatile organic solvents, such as nonafluoromethoxybutane or perfluoromethylcyclopentane, may also be used.

According to one embodiment of the compositions according to the present disclosure, the water-insoluble volatile compound that is liquid at room temperature is chosen from volatile hydrocarbon-based oils comprising from 8 to 16 carbon atoms, and mixtures thereof.

The non-aqueous solvent phase may also comprise at least one water-insoluble non-volatile compound that is liquid at room temperature, such as at least one non-volatile oil, which may be chosen from, for example, non-volatile hydrocarbon-based oils, silicone oils, fluoro oils, and mixtures thereof.

Among non-volatile hydrocarbon-based oils that may be used according to the present disclosure, non limiting mention may be made of:

hydrocarbon-based oils of plant origin, such as triglycerides comprising fatty acid esters of glycerol, the fatty acids of which may have chain lengths ranging from C₄ to C₂₄, these chains being linear or branched, and saturated or unsaturated; these oils include, for example, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil and musk rose oil; or alternatively caprylic/capric acid triglycerides, for instance those sold by the company Stearineries Dubois or those sold under the names “Miglyol 810®”, “812®” and “818®” by the company Dynamit Nobel,

synthetic ethers comprising from 10 to 40 carbon atoms;

linear or branched hydrocarbons of mineral or synthetic origin, such as petroleum jelly, polydecenes, hydrogenated polyisobutene such as parleam, and squalane, and mixtures thereof;

synthetic esters, for instance oils of formula R₁COOR₂ wherein R₁ is chosen from linear or branched fatty acid residues comprising from 1 to 40 carbon atoms and R₂ is chosen from hydrocarbon-based chains, such as, for example, a branched chain, comprising from 1 to 40 carbon atoms, on condition that R₁+R₂≧10 carbon atoms, for instance purcellin oil (cetostearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, and alcohol or polyalcohol octanoates, decanoates or ricinoleates, for instance propylene glycol dioctanoate; hydroxylated esters, for instance isostearyl lactate or diisostearyl malate; and pentaerythritol esters;

fatty alcohols that are liquid at room temperature, with a branched and/or unsaturated carbon-based chain comprising from 12 to 26 carbon atoms, for instance octyldodecanol, isostearyl alcohol, oleyl alcohol, 2-hexyldecanol, 2-butyloctanol or 2-undecylpentadecanol;

higher fatty acids such as oleic acid, linoleic acid and linolenic acid; and mixtures thereof.

Among non-volatile silicone oils that may be used in the composition according to the disclosure, non-limiting mention may be made of non-volatile polydimethylsiloxanes (PDMSs), polydimethylsiloxanes comprising alkyl or alkoxy groups, which are pendent and/or at the end of a silicone chain, these groups each comprising from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates.

Among fluoro oils that may be used in the composition of the present disclosure, non-limiting mention may be made of fluorosilicone oils, fluoro polyethers and fluorosilicones, such as those described in European Patent 847 752.

The amount of water-insoluble non-volatile compound that is liquid at room temperature may range from, for example, 0.01% to 30% by weight, such as from 0.1% to 25%, by weight relative to the total weight of the composition.

Water and/or Water-Soluble Solvent

The compositions according to the present disclosure comprise water and/or at least one water-soluble solvent.

In the present disclosure, the term “water-soluble solvent” denotes a compound that is liquid at room temperature and miscible with water (water miscibility of greater than 50% by weight at 25° C. and atmospheric pressure).

The water-soluble solvents that may be used in the compositions according to the present disclosure may also be volatile.

Among the water-soluble solvents that may be used in the compositions according to the present disclosure, non-limiting mention may be made of lower monoalcohols comprising from 1 to 5 carbon atoms, such as ethanol and isopropanol, glycols comprising from 2 to 8 carbon atoms, such as ethylene glycol, propylene glycol, 1,3-butylene glycol and dipropylene glycol, C₃ and C₄ ketones and C₂-C₄ aldehydes.

The water and/or the at least one water-soluble solvent may be introduced as such into the formulation or may be incorporated therein by means of one or more ingredients constituting the composition. For example, water may be introduced into the composition by means of the introduction of latex or pseudolatex, i.e., an aqueous dispersion of polymer particles.

According to one embodiment, the amount of water and/or at least one water-soluble solvent in the composition may be less than or equal to 20% by weight, such as, for example, less than or equal to 10% by weight, less than or equal to 5% by weight or less than 2% by weight relative to the total weight of the composition.

Wax

The waxes that may be used in compositions of the present disclosure may be lipophilic compounds that are solid at room temperature (25° C.), with a solid/liquid reversible change of state, having a melting point of greater than or equal to 30° C., which may be up to 120° C.

By bringing the wax to the liquid form (melting), it is possible to make it miscible with oils and to form a microscopically uniform mixture, but on bringing the mixture back to room temperature, recrystallization of the wax in the oils of the mixture may be obtained.

In at least one embodiment, the waxes may have a melting point of greater than about 45° C., such as greater than or equal to 50° C. or greater than or equal to 55° C.

The melting point of the wax may be measured using a differential scanning calorimeter (DSC), for example the calorimeter sold under the name DSC 30 by the company Mettler.

The measuring protocol is as follows:

A sample of 15 mg of product placed in a crucible is subjected to a first temperature rise ranging from 0° C. to 120° C., at a heating rate of 10° C./minute. It is then cooled from 120° C. to 0° C. at a cooling rate of 10° C./minute and is finally subjected to a second temperature increase ranging from 0° C. to 120° C. at a heating rate of 5° C./minute. During the second temperature increase, the variation of the difference in power absorbed by the empty crucible and by the crucible containing the sample of product is measured as a function of the temperature. The melting point of the compound is the temperature value corresponding to the top of the peak of the curve representing the variation in the difference in absorbed power as a function of the temperature.

The wax may have a hardness ranging from 0.05 MPa to 30 MPa, such as a hardness ranging from 6 MPa to 15 MPa. The hardness may be determined by measuring the compression force, measured at 20° C. using the texturometer sold under the name TA-XT2i by the company Rheo, equipped with a stainless-steel cylindrical spindle 2 mm in diameter, travelling at a measuring speed of 0.1 mm/s, and penetrating into the wax to a penetration depth of 0.3 mm.

The measuring protocol is as follows:

The wax is melted at a temperature equal to the melting point of the wax+20° C. The molten wax is poured into a container 30 mm in diameter and 20 mm deep. The wax is recrystallized at room temperature (25° C.) for 24 hours and is then stored for at least 1 hour at 20° C., before performing the hardness measurement. The hardness value is the maximum compression force measured, divided by the area of the texturometer spindle in contact with the wax.

The waxes may be chosen from, for example, waxes of animal, plant, mineral or synthetic origin, and mixtures thereof, which are solid and rigid at room temperature. Among examples of waxes that may be used according to the present disclosure, non-limiting mention may be hydrocarbon-based waxes, for instance beeswax, lanolin wax and Chinese insect waxes; rice wax, carnauba wax, candelilla wax, ouricurry wax, esparto grass wax, cork fiber wax, sugar cane wax, Japan wax and sumach wax; montan wax, microcrystalline waxes, paraffins and ozokerite; polyethylene waxes, the waxes obtained by Fischer-Tropsch synthesis and waxy copolymers, and also esters thereof.

According to one embodiment, the composition is wax-free. The term “wax-free” means a composition comprising less than 10% by weight, such as less than 7%, less than 5% or less than 3% by weight of wax relative to the total weight of the composition. In further embodiments, the composition is totally wax-free.

Film-Forming Polymer

The composition according to the present disclosure may also comprise at least one liposoluble film-forming polymer (i.e., a polymer that is soluble in a liquid fatty phase comprising organic solvents or oils such as those described above) or lipophilic film-forming polymer (i.e. a polymer that is compatible with a liquid fatty phase comprising organic solvents or oils such as those described above), which is also referred to as the at least one “second polymer”.

For the purposes of the present disclosure, the expression “liquid fatty phase” means a fatty phase which is liquid at room temperature (25° C.) and atmospheric pressure (760 mmHg, i.e. 10⁵ Pa), composed of one or more fatty substances that are liquid at room temperature, such as the oils described above, which are generally mutually compatible, which generally corresponds to the non-aqueous solvent phase of the composition.

In the present disclosure, the term “film-forming polymer” means a polymer capable of forming, by itself or in the presence of an auxiliary film-forming agent, a continuous film that adheres to a support, such as, for example, keratin materials.

Examples of liposoluble polymers that may be mentioned include copolymers of vinyl ester (the vinyl group being directly linked to the oxygen atom of the ester group and the vinyl ester comprising a saturated, linear or branched hydrocarbon-based radical of 1 to 19 carbon atoms, linked to the carbonyl of the ester group) and of at least one other monomer, which may be a vinyl ester (other than the vinyl ester already present), an α-olefin (comprising from 8 to 28 carbon atoms), an alkyl vinyl ether (in which the alkyl group comprises from 2 to 18 carbon atoms) or an allylic or methallylic ester (comprising a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers that may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Examples of liposoluble film-forming polymers that may also be mentioned include liposoluble copolymers, and in particular those resulting from the copolymerization of vinyl esters comprising from 9 to 22 carbon atoms or of alkyl acrylates or methacrylates, and alkyl radicals comprising from 10 to 20 carbon atoms.

Such liposoluble copolymers may be chosen from polyvinyl stearate, polyvinyl stearate crosslinked with the aid of divinylbenzene, of diallyl ether or of diallyl phthalate, polystearyl(meth)acrylate, polyvinyl laurate and polylauryl(meth)acrylate, these poly(meth)acrylates optionally crosslinked with the aid of ethylene glycol dimethacrylate or tetraethylene glycol dimethacrylate.

The liposoluble copolymers defined above are known and are described, for example, in French Patent Application No. FR-A-2 232 303; they may have a weight-average molecular weight ranging from 2000 to 500,000, such as, for example, from 4000 to 200,000.

As liposoluble film-forming polymers that may be used in the composition of the present disclosure, non-limiting mention may also be made of polyalkylenes including copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and copolymers of vinylpyrrolidone and of C₂ to C₄₀ or C₃ to C₂₀ alkene. As examples of VP copolymers which may be used in the composition, non-limiting mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Non-limiting mention may also be made of silicone resins, which may be soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing a type of unit.

Examples of commercially available polymethylsilsesquioxane resins that may be mentioned include those sold:

by the company Wacker under the reference Resin MK, such as Belsil PMS MK;

by the company Shin-Etsu under the reference KR-220L.

Siloxysilicate resins that may be mentioned include trimethyl siloxysilicate (TMS) resins such as those sold under the reference SR 1000 by the company General Electric or under the reference TMS 803 by the company Wacker. Mention may also be made of the trimethyl siloxysilicate resins sold in a solvent such as cyclomethicone, sold under the name “KF-7312J” by the company Shin-Etsu, and “DC 749” and “DC 593” by the company Dow Corning.

Non-limiting mention may also be made of copolymers of silicone resins such as those mentioned above with polydimethylsiloxanes, for instance the pressure-sensitive adhesive copolymers sold by the company Dow Corning under the reference Bio-PSA and described in U.S. Pat. No. 5,162,410, or the silicone copolymers derived from the reaction of a silicone resin, such as those described above, and a diorganosiloxane as described in International Patent Application Publication No. WO 2004/073,626.

Silicone polyamides of the polyorganosiloxane type may also be used, such as those described in U.S. Pat. Nos. 5,874,069; 5,919,441; 6,051,216; and 5,981,680.

These silicone polymers may belong to the following two families:

polyorganosiloxanes comprising at least two groups capable of establishing hydrogen interactions, these two groups being located in the polymer chain, and/or

polyorganosiloxanes comprising at least two groups capable of establishing hydrogen interactions, these two groups being located on grafts or branches.

According to one embodiment of the invention, the film-forming polymer is a film-forming linear block ethylenic polymer, which may comprise at least one first block and at least one second block with different glass transition temperatures (Tg), the first and second blocks being linked together via an intermediate block comprising at least one constituent monomer of the first block and at least one constituent monomer of the second block.

The first and second blocks of the block polymer may be mutually incompatible.

Such polymers are described, for example, in European Patent No. EP 1 411 069 or International Patent Application Publication No. WO 04/028,488.

The lipophilic or liposoluble film-forming polymer may also be present in the composition in the form of particles dispersed in a non-aqueous solvent phase. The techniques for preparing these dispersions are well known to those skilled in the art.

As examples of non-aqueous dispersions of film-forming polymer, non-limiting mention may be made of the dispersions described, for example, in European Patent No. EP 749 746 and acrylic polymer particles, surface-stabilized with a stabilizer, as a dispersion in a fatty phase (for example isododecane), for instance Mexomer PAP® from the company Chimex, dispersions of particles of a grafted ethylenic polymer, such as an acrylic polymer, in a liquid fatty phase, the ethylenic polymer being dispersed in the absence of additional stabilizer at the surface of the particles, as described in International Patent Application Publication No. WO 04/055081.

The second polymer chosen from liposoluble or lipophilic film-forming polymers may be present in a solids content ranging from 0.1% to 40% by weight, such as from 0.5% to 30% by weight or from 1% to 20% by weight relative to the total weight of the composition.

According to one embodiment, the composition according to the present disclosure comprises at least one lipophilic or liposoluble polymer in a polymer solids content of greater than or equal to 10% by weight, such as greater than or equal to 17% by weight or greater than or equal to 20% by weight relative to the total weight of the composition.

The composition may also comprise an additional film-forming polymer chosen, for example, from hydrophilic film-forming polymers such as:

proteins, for instance proteins of plant origin such as wheat proteins and soybean proteins; proteins of animal origin such as keratins, for example keratin hydrolysates and sulfonic keratins;

anionic, cationic, amphoteric or nonionic chitin or chitosan polymers;

polymers of cellulose such as hydroxyethylcellulose, hydroxypropylcellulose, methylcellulose, ethylhydroxyethylcellulose and carboxymethylcellulose, and quaternized cellulose derivatives;

vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;

polymers of natural origin, which are optionally modified, such as:
gum arabics, guar gum, xanthan derivatives and karaya gum;
alginates and carrageenans;
glycoaminoglycans, and hyaluronic acid and derivatives thereof;
shellac resin, sandarac gum, dammar resins, elemi gums and copal resins;
deoxyribonucleic acid;
mucopolysaccharides such as hyaluronic acid and chondroitin sulfate, and mixtures thereof.

The additional film-forming polymer may also be in the form of an aqueous dispersion, for instance the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® by the company Interpolymer, or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer; the sulfopolyesters sold under the brand name “Eastman AQ®” by the company Eastman Chemical Products, vinyl dispersions, for instance Mexomer PAM® from the company Chimex, and mixtures thereof.

The additional film-forming polymer may be present in a solids content ranging from 0.1% to 30% by weight, such as from 0.5% to 20% by weight or from 1% to 15% by weight relative to the total weight of the composition.

The composition according to the present disclosure may comprise a plasticizer that promotes the formation of a film with the film-forming polymer. Such a plasticizer may be chosen from any compound known to those skilled in the art as being capable of fulfilling the desired function.

Dyestuff

The composition according to the present disclosure may also comprise at least one dyestuff, for instance pulverulent dyes, liposoluble dyes and water-soluble dyes.

The pulverulent dyestuffs may be chosen from pigments and nacres.

The pigments may be white or colored, mineral and/or organic, and coated or uncoated. Among the mineral pigments that may be used according to the present disclosure, non-limiting mention may be made of titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be used, non-limiting mention may be made of carbon black, pigments of D & C type, and lakes based on cochineal carmine or on barium, strontium, calcium or aluminium.

The nacres may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

These dyestuffs may be present in an amount ranging from 0.01% to 30% by weight relative to the total weight of the composition.

According to one embodiment, the composition is free of dyestuff.

Fillers

The composition according to the present disclosure may also comprise at least one filler.

The fillers may be chosen from those that are well known to persons skilled in the art and commonly used in cosmetic compositions. The fillers may be mineral or organic, and lamellar or spherical. Non-limiting mention may be made of talc, mica, silica, kaolin, polyamide powders, for instance the Nylon® sold under the trade name Orgasol® by the company Atochem, poly-β-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders, such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms, such as, for example, from 12 to 18 carbon atoms, for example zinc, magnesium or lithium stearate, zinc laurate and magnesium myristate.

The fillers may be present in an amount ranging from 0.1% to 25% such as from 1% to 20% by weight relative to the total weight of the composition.

The composition of the present disclosure may also comprise any cosmetically acceptable additives chosen from those usually used in cosmetics, such as, for example, antioxidants, preserving agents, fragrances, neutralizers, plasticizers, thickeners or gelling agents, fibers and cosmetic active agents, and mixtures thereof.

The composition may also comprise, besides the first polymer defined above, at least one lipophilic gelling agent that may be organic or mineral, and polymeric or molecular.

Among mineral lipophilic gelling agents that may be used according to the present disclosure, non-limiting mention may be made of optionally modified clays, for instance hectorites modified with a C₁₀ to C₂₂ fatty acid ammonium chloride, for instance hectorite modified with distearyldimethylammonium chloride, for instance the product sold under the name “Bentone 38V®” by the company Elementis.

Non-limiting mention may also be made of fumed silica optionally subjected to a hydrophobic surface treatment, the particle size of which is less than 1 μm. For example, the surface of the silica may be chemically modified by chemical reaction generating a reduced number of silanol groups present at the surface of the silica. Silanol groups may also be substituted with hydrophobic groups to obtain a hydrophobic silica. The hydrophobic groups may be:

trimethylsiloxyl groups, which are obtained, for example, by treating fumed silica in the presence of hexamethyldisilazane. Silicas thus treated are known as “silica silylate” according to the CTFA (6th edition, 1995). They are sold, for example, under the names “Aerosil R812®” by the company Degussa, and “Cab-O-Sil TS-530®” by the company Cabot;

dimethylsilyloxyl or polydimethylsiloxane groups, which are obtained, for example, by treating fumed silica in the presence of polydimethylsiloxane or dimethyldichlorosilane. Silicas thus treated are known as “silica dimethyl silylate” according to the CTFA (6th edition, 1995). They are sold, for example, under the names “Aerosil R972®” and “Aerosil R974®” by the company Degussa, and “Cab-O-Sil TS-610®” and “Cab-O-Sil TS-720®” by the company Cabot.

The hydrophobic fumed silica preferably has a particle size that may be nanometric to micrometric, for example ranging from about 5 to 200 nm.

The polymeric organic lipophilic gelling agents are, for example, partially or totally crosslinked elastomeric organopolysiloxanes of three-dimensional structure, for instance those sold under the names KSG6®, KSG16® and KSG18® from Shin-Etsu, Trefil E-505C® or Trefil E-506C® from Dow Corning, Gransil SR-CYC®, SR DMF 10®, SR-DC556®, SR 5CYC gel®, SR DMF 10 gel® and SR DC 556 gel® from Grant Industries and SF 1204® and JK 113® from General Electric; ethylcellulose, for instance the product sold under the name Ethocel by Dow Chemical; galactomannans comprising from one to six such as, for example, from two to four hydroxyl groups per saccharide, substituted with a saturated or unsaturated alkyl chain, for instance guar gum alkylated with C₁ to C₆, such as, for example, C₁ to C₃, alkyl chains, and mixtures thereof. Block copolymers of “diblock” or “triblock” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Shell Chemical Co., or of the polystyrene/copoly(ethylene-butylene) type.

Among the gelling agents that may be used in the compositions according to the present disclosure, non-limiting mention may also be made of fatty acid esters of dextrin, such as dextrin palmitates, including the products sold under the name Rheopearl TL® or Rheopearl KL® by the company Chiba Flour.

The composition according to the disclosure may also comprise fibers to allow an improvement in the lengthening effect.

The term “fiber” should be understood as meaning an object of length L and diameter D such that L is very much greater than D, D being the diameter of the circle in which the cross section of the fiber is inscribed. For example, the ratio L/D (or form factor) is chosen in the range from 3.5 to 2500, such as, for example, from 5 to 500 or from 5 to 150.

The fibers may have a length ranging from 1 μm to 10 mm, such as from 0.1 mm to 5 mm or from 0.3 mm to 3 mm.

The fibers that may be used in the composition may be chosen from rigid or non-rigid fibers, and may be of synthetic or natural, mineral or organic origin.

Among fibers that may be used in the composition according to the present disclosure, non-limiting mention may be made of non-rigid fibers such as polyamide (Nylon®) fibers or rigid fibers such as polyimideamide fibers, for instance those sold under the names Kermel and Kermel Tech by the company Rhodia or poly(p-phenylenetereph-thalamide) (or aramid) fibers sold, for example, under the name Kevlar® by the company DuPont de Nemours.

Among cosmetic active agents that may be used in the compositions according to the present disclosure, non-limiting mention may be made of emollients, moisturizers, vitamins and screening agents, such as sunscreens.

Needless to say, a person skilled in the art will take care to select the optional additional additives and/or the amount thereof such that the beneficial properties of the composition according to the present disclosure are not, or are not substantially, adversely affected by the envisaged addition.

The compositions of the present disclosure may, for example, be applied to the eyelashes, using a brush, a comb-brush or a comb as described, for example, in U.S. Pat. No. 6,581,610, U.S. Pat. No. 6,343,607, U.S. Pat. No. 6,412,469, U.S. Pat. No. 6,546,937, EP 1 157 632, WO 01/05274, WO 01/05272, WO 01/05271, EP 1 306 029 and EP 1 342 428, the content of which is hereby incorporated into the present disclosure by reference.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the invention as approximations, the numerical values set forth in the specific examples are reported as precisely as possible. Any numerical value, however, inherently contains certain errors necessarily resulting from the standard deviation found in its respective testing measurement.

The examples that follow serve to illustrate the invention without, however, being limiting in nature.

### EXAMPLE 1

A waterproof mascara having the composition below was prepared:

| | |
|---|---|
| Dispersion of poly(methyl methacrylate/acrylic acid) | 6% |
| particles surface-stabilized in isododecane with a | |
| polystyrene/copoly(ethylene-propylene) diblock | |
| copolymer (Kraton G1701), at a polymer solids | |
| content of 24.5% (Mexomer PAP from Chimex) | |
| Vinylpyrrolidone/1-eicosene copolymer | 8% |
| (“Antaron V 220 ®” from ISP) | |
| Polyvinyl laurate (“Mexomer PP ®” from Chimex) | 0.9% |
| Allyl stearate/vinyl acetate copolymer | 8.1% |
| (“Mexomer PQ ®” from Chimex) | |
| Ethylenediamine/stearyl dilinoleate copolymer | 3% |
| (UNICLEAR from the company Arizona Chemical) | |
| Modified hectorite (“Bentone 38V ®” from Elementis) | 7.5% |
| Preserving agents | qs |
| Isododecane | qs 100% |

This mascara was applied to eyelashes precoated with a base coat of a conventional emulsion mascara that had been allowed to dry. An eyelash makeup result that had good staying power and that was resistant to water and/or sebum was thus obtained.

### EXAMPLE 2

The cream mascara below was prepared:

| | |
|---|---|
| Carnauba wax | 2.9% |
| Beeswax | 3.62% |
| Paraffin wax | 11.45% |
| Hydroxyethyl cellulose quaternized with 2,3- | 0.1% |
| epoxypropyltrimethylammonium chloride | |
| Hydroxyethyl cellulose | 0.91% |
| Unstabilized sodium polymethacrylate at 25% in water | 1% |
| (Darvan 7 from Vanderbilt) | |
| Gum arabic | 3.45% |
| Mixture of polydimethylsiloxane and of hydrated silica | 0.13% |
| Triethanolamine | 2.4% |
| Stearic acid | 5.82% |
| D-Panthenol | 0.5% |
| Aminomethylpropanediol | 0.5% |
| Pigments | 5.45% |
| Preserving agents | qs |
| Water | qs 100% |

A first coat of this mascara was applied to the eyelashes and allowed to dry for a few seconds, and a second coat of mascara according to Example 1 was then applied over the first coat. A film of waterproof makeup that showed good staying power was obtained on the eyelashes.

What is claimed is:

1. A composition for coating keratin fibers, comprising, in a cosmetically acceptable medium:
a non-aqueous solvent phase,
at least one first polymer comprising
a) a polymer skeleton having hydrocarbon-based repeating units comprising at least one heteroatom, and
optionally b) at least one pendent fatty chain and/or at least one terminal fatty chain, which are optionally functionalized, comprising at least 4 carbon atoms and linked to the hydrocarbon-based units,
the composition being wax-free and comprising less than 20% of water and/or water-soluble solvent.

2. The composition according to claim 1, further comprising at least one second polymer chosen from liposoluble or lipophilic film-forming polymers.

3. The composition according to claim 2, wherein the at least one second polymer chosen from liposoluble or lipophilic film-forming polymers is chosen from vinyl ester polymers and copolymers, vinylpyrrolidone copolymers, and dispersions of acrylic polymer particles in a liquid fatty phase, and mixtures thereof.

4. The composition according to claim 2, wherein the at least one second polymer chosen from liposoluble or lipophilic film-forming polymers is present in a solids content ranging from 0.1% to 40% by weight relative to the total weight of the composition.

5. The composition according to claim 4, wherein the at least one second polymer chosen from liposoluble or lipophilic film-forming polymers is present in a solids content ranging from 1% to 20% by weight relative to the total weight of the composition.

6. The composition according to claim 1, wherein the hydrocarbon-based repeating units comprise a nitrogen atom.

7. The composition according to claim 1, wherein the hydrocarbon-based repeating units are amide groups.

8. The composition according to claim 7, wherein the pendent fatty chains are directly linked to at least one of the nitrogen atoms of the amide groups.

9. The composition according to claim 1, wherein the fatty chains are present in an amount ranging from 40% to 98% of the total number of units comprising a hetero atom and of fatty chains.

10. The composition according to claim 9, wherein the fatty chains are present in an amount ranging from 50% to 95% of the total number of units comprising a hetero atom and of fatty chains.

11. The composition according to claim 1, wherein the pendent fatty chains are directly linked to at least one of the heteroatoms.

12. The composition according to claim 1, wherein the fatty chains contain from 6 to 120 carbon atoms.

13. The composition according to claim 12, wherein the fatty chains contain from 8 to 120 carbon atoms.

14. The composition according to claim 12, wherein the fatty chains are present in an amount ranging from 40% to 98% of the total number of amide units and of fatty chains.

15. The composition according to claim 14, wherein the fatty chains are present in an amount ranging from 50% to 95% of the total number of amide units and of fatty chains.

16. The composition according to claim 1, wherein the average molar mass of the at least one first polymer is less than 100,000.

17. The composition according to claim 1, wherein the at least one terminal fatty chain is linked to the polymer skeleton via bonding groups.

18. The composition according to claim 17, wherein the bonding groups are ester groups.

19. The composition according to claim 18, wherein the fatty chains contain from 12 to 68 carbon atoms.

20. The composition according to claim 1, wherein the at least one first polymer is chosen from polyamides of formula (II): 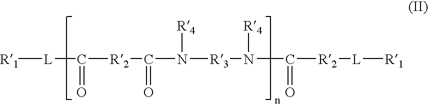
wherein:
n is an integer ranging from 1 to 30;
R′₁, which are the same or different, are fatty chains chosen from alkyl and alkenyl groups comprising at least 1 carbon atom;
R′₂, which are the same or different, are chosen from hydrocarbon-based radicals comprising from 1 to 52 carbon atoms;
R′₃, which are the same or different, are chosen from organic groups comprising at least one atom chosen from carbon, hydrogen and nitrogen atoms, with the proviso that R′₃ comprises at least 3 carbon atoms;
R′₄, which are the same or different, are chosen from a hydrogen atom, an alkyl group comprising from 1 to 10 carbon atoms, and a direct bond to at least one group chosen from R′₃ and another R′₄ such that the nitrogen atom to which both R′₃ and R′₄ are attached forms part of a heterocyclic structure defined by R′₄—N—R′₃, with the proviso that at least 50% of the R′₄ groups are hydrogen atoms; and
L is a bonding group chosen from ester, ether, amine, urea, urethane, thioester, thioether, thiourea and thiourethane groups, optionally substituted with at least one group R′₁ as defined above.

21. The composition according to claim 20, wherein R′₁ are fatty chains chosen from alkyl and alkenyl groups comprising from 4 to 24 carbon atoms.

22. The composition according to claim 1, wherein the at least one first polymer is chosen from polyamides of formula (I) below: 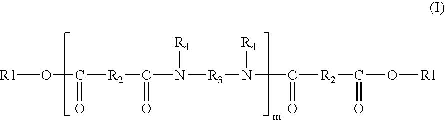
wherein:
m is a whole number of amide units such that the number of ester groups represents from 10% to 50% of the total number of ester and amide groups;
R₁, which are the same or different, are chosen from alkyl and alkenyl groups comprising at least 4 carbon atoms;
R₂, which are the same or different, are chosen from C₄ to C₄₂ hydrocarbon-based groups, with the proviso that 50% of the groups R₂ are chosen from C₃₀ to C₄₂ hydrocarbon-based groups;
R₃, which are the same or different, are chosen from organic groups comprising at least 2 carbon atoms, hydrogen atoms and optionally at least one oxygen or nitrogen atoms; and
R₄, which are the same or different, are chosen from a hydrogen atom, C₁ to C₁₀ alkyl groups and a direct bond to an R₃ group or to another R₄ group, such that the nitrogen atom to which both R₃ and R₄ are attached forms part of a heterocyclic structure defined by R₄—N—R₃, with the proviso that at least 50% of the groups R₄ are hydrogen atoms.

23. The composition according to claim 22, wherein R₁ are chosen from are chosen from alkyl and alkenyl groups comprising from 4 to 24 carbon atoms.

24. The composition according to claim 1, wherein the at least one first polymer is present in an amount ranging from 0.01% to 20% by weight relative to the total weight of the composition.

25. The composition according to claim 1, wherein the non-aqueous solvent phase comprises at least one volatile compound.

26. The composition according to claim 1, further comprising at least one additive chosen from dyestuffs, antioxidants, fillers, pasty fatty substances, preserving agents, fragrances, neutralizers, gelling agents, thickeners, vitamins, coalescers and plasticizers, and mixtures thereof.

27. A composition for coating keratin fibers, comprising, in a cosmetically acceptable medium:
a non-aqueous solvent phase,
at least one first polymer comprising
a) a polymer skeleton having hydrocarbon-based repeating units comprising at least one hetero atom, and
optionally b) at least one pendent fatty chain and/or at least one terminal fatty chain, which are optionally functionalized, comprising at least 4 carbon atoms and linked to the hydrocarbon-based units,
the composition having a solids content of less than or equal to 37% by weight relative to the total weight of the composition.

28. The composition according to claim 27, further comprising at least one second polymer chosen from liposoluble or lipophilic film-forming polymers.

29. The composition according to claim 28, wherein the at least one second polymer chosen from liposoluble or lipophilic film-forming polymers is chosen from vinyl ester polymers and copolymers, vinylpyrrolidone copolymers, and dispersions of acrylic polymer particles in a liquid fatty phase, and mixtures thereof.

30. The composition according to claim 28, wherein the at least one second polymer chosen from liposoluble or lipophilic film-forming polymers is present in a solids content ranging from 0.1% to 40% by weight relative to the total weight of the composition.

31. The composition according to claim 27, wherein the hydrocarbon-based repeating units comprise a nitrogen atom.

32. The composition according to claim 27, wherein the hydrocarbon-based repeating units are amide groups.

33. The composition according to claim 32, wherein the pendent fatty chains are directly linked to at least one of the nitrogen atoms of the amide groups.

34. The composition according to claim 27, wherein the fatty chains are present in an amount ranging from 40% to 98% of the total number of units comprising a hetero atom and of fatty chains.

35. The composition according to claim 27, wherein the pendent fatty chains are directly linked to at least one of the heteroatoms.

36. The composition according to claim 27, wherein the fatty chains contain from 6 to 120 carbon atoms.

37. The composition according to claim 27, wherein the fatty chains are present in an amount ranging from 40% to 98% of the total number of amide units and of fatty chains.

38. The composition according to claim 27, wherein the average molar mass of the at least one first polymer is less than 100,000.

39. The composition according to claim 27, wherein the at least one terminal fatty chain is linked to the polymer skeleton via bonding groups.

40. The composition according to claim 39, wherein the bonding groups are ester groups.

41. The composition according to claim 27, wherein the fatty chains contain from 12 to 68 carbon atoms.

42. The composition according to claim 27, wherein the at least one first polymer is chosen from polyamides of formula (II): 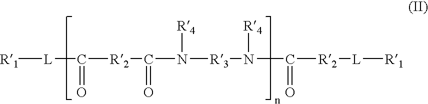
wherein:
n is an integer ranging from 1 to 30;
R′₁, which are the same or different, are fatty chains chosen from alkyl and alkenyl groups comprising at least 1 carbon atom;
R′₂, which are the same or different, are chosen from hydrocarbon-based radicals comprising from 1 to 52 carbon atoms;
R′₃, which are the same or different, are chosen from organic groups comprising at least one atom chosen from carbon, hydrogen and nitrogen atoms, with the proviso that R′₃ comprises at least 3 carbon atoms;
R′₄, which are the same or different, are chosen from a hydrogen atom, an alkyl group comprising from 1 to 10 carbon atoms, and a direct bond to at least one group chosen from R′₃ and another R′₄ such that the nitrogen atom to which both R′₃ and R′₄ are attached forms part of a heterocyclic structure defined by R′₄—N—R′₃, with the proviso that at least 50% of the R′₄ groups are hydrogen atoms; and
L is a bonding group chosen from ester, ether, amine, urea, urethane, thioester, thioether, thiourea and thiourethane groups, optionally substituted with at least one group R′₁ as defined above.

43. The composition according to claim 42, wherein R′₁ are fatty chains chosen from alkyl and alkenyl groups comprising from 4 to 24 carbon atoms.

44. The composition according to claim 27, wherein the at least one first polymer is chosen from polyamides of formula (I) below: 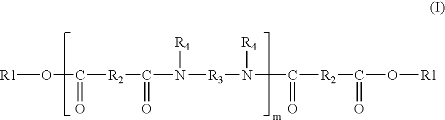
wherein:
m is a whole number of amide units such that the number of ester groups represents from 10% to 50% of the total number of ester and amide groups;
R₁, which are the same or different, are chosen from alkyl and alkenyl groups comprising at least 4 carbon atoms;
R₂, which are the same or different, are chosen from C₄ to C₄₂ hydrocarbon-based groups, with the proviso that 50% of the groups R₂ are chosen from C₃₀ to C₄₂ hydrocarbon-based groups;
R₃, which are the same or different, are chosen from organic groups comprising at least 2 carbon atoms, hydrogen atoms and optionally at least one oxygen or nitrogen atoms; and
R₄, which are the same or different, are chosen from a hydrogen atom, C₁ to C₁₀ alkyl groups and a direct bond to an R₃ group or to another R₄ group, such that the nitrogen atom to which both R₃ and R₄ are attached forms part of a heterocyclic structure defined by R₄—N—R₃, with the proviso that at least 50% of the groups R₄ are hydrogen atoms.

45. The composition according to claim 44, wherein R₁ are chosen from alkyl and alkenyl groups comprising from 4 to 24 carbon atoms.

46. A composition for coating keratin fibers, comprising, in a cosmetically acceptable medium:
a non-aqueous solvent phase,
at least one first polymer comprising
a) a polymer skeleton having hydrocarbon-based repeating units comprising at least one heteroatom, and
optionally b) at least one pendent fatty chain and/or at least one terminal fatty chain, which are optionally functionalized, comprising at least 4 carbon atoms and linked to the hydrocarbon-based units,
at least one second polymer chosen from liposoluble or lipophilic film-forming polymers in a solids content of greater than or equal to 10% by weight relative to the total weight of the composition.

47. The composition according to claim 46, wherein the at least one second polymer chosen from liposoluble or lipophilic film-forming polymers is chosen from vinyl ester polymers and copolymers, vinylpyrrolidone copolymers, and dispersions of acrylic polymer particles in a liquid fatty phase, and mixtures thereof.

48. The composition according to claim 46, wherein the at least one second polymer chosen from liposoluble or lipophilic film-forming polymers is present in a solids content of greater than or equal to 17% by weight relative to the total weight of the composition.

49. The composition according to claim 46, wherein the at least one first polymer is chosen from polyamides of formula (II): 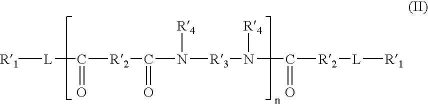
wherein:
n is an integer ranging from 1 to 30;
R′₁, which are the same or different, are fatty chains chosen from alkyl and alkenyl groups comprising at least 1 carbon atom;
R′₂, which are the same or different, are chosen from hydrocarbon-based radicals comprising from 1 to 52 carbon atoms;
R′₃, which are the same or different, are chosen from organic groups comprising at least one atom chosen from carbon, hydrogen and nitrogen atoms, with the proviso that R′₃ comprises at least 3 carbon atoms;
R′₄, which are the same or different, are chosen from a hydrogen atom, an alkyl group comprising from 1 to 10 carbon atoms, and a direct bond to at least one group chosen from R′₃ and another R′₄ such that the nitrogen atom to which both R′₃ and R′₄ are attached forms part of a heterocyclic structure defined by R′₄—N—R′₃, with the proviso that at least 50% of the R′₄ groups are hydrogen atoms; and
L is a bonding group chosen from ester, ether, amine, urea, urethane, thioester, thioether, thiourea and thiourethane groups, optionally substituted with at least one group R′₁ as defined above.

50. The composition according to claim 46, wherein the at least one first polymer is chosen from polyamides of formula (I) below: 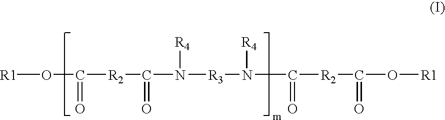
wherein:
m is a whole number of amide units such that the number of ester groups represents from 10% to 50% of the total number of ester and amide groups;
R₁, which are the same or different, are chosen from alkyl and alkenyl groups comprising at least 4 carbon atoms;
R₂, which are the same or different, are chosen from C₄ to C₄₂ hydrocarbon-based groups, with the proviso that 50% of the groups R₂ are chosen from C₃₀ to C₄₂ hydrocarbon-based groups;
R₃, which are the same or different, are chosen from organic groups comprising at least 2 carbon atoms, hydrogen atoms and optionally at least one oxygen or nitrogen atoms; and
R₄, which are the same or different, are chosen from a hydrogen atom, C₁ to C₁₀ alkyl groups and a direct bond to an R₃ group or to another R₄ group, such that the nitrogen atom to which both R₃ and R₄ are attached forms part of a heterocyclic structure defined by R₄—N—R₃, with the proviso that at least 50% of the groups R₄ are hydrogen atoms.

51. A process for making up or for the non-therapeutic care of keratin fibers, comprising applying to the keratin fibers a composition comprising, in a cosmetically acceptable medium:
a non-aqueous solvent phase,
at least one first polymer comprising
a) a polymer skeleton, having hydrocarbon-based repeating units comprising at least one heteroatom, and
optionally b) at least one pendent fatty chain and/or at least one terminal fatty chain, which are optionally functionalized, comprising at least 4 carbon atoms and linked to the hydrocarbon-based units,
the composition being wax-free and comprising less than 20% of water and/or water-soluble solvent.

52. A process for coating keratin fibers, comprising applying to the keratin fibers:
i) a first coat of a first composition,
ii) and then, after partial or total drying of the first coat, at least one second coat of a second composition comprising
a non-aqueous solvent phase,
at least one first polymer comprising
a) a polymer skeleton, having hydrocarbon-based repeating units comprising at least one heteroatom, and
optionally b) at least one pendent fatty chain and/or at least one terminal fatty chain, which are optionally functionalized, comprising at least 4 carbon atoms and linked to the hydrocarbon-based units,
the second composition being wax-free.

53. The process according to claim 52, wherein the keratin fibers are eyelashes.

54. A process for coating keratin fibers, comprising applying to the keratin fibers:
i) at least one first coat of a first composition comprising at least 30% by weight water and/or a water-soluble solvent,
ii) and then, after partial or total drying of the at least one first coat, at least one second coat of a second composition comprising
a non-aqueous solvent phase,
at least one first polymer comprising
a) a polymer skeleton, having hydrocarbon-based repeating units comprising at least one heteroatom, and
optionally b) at least one pendent fatty chain and/or at least one terminal fatty chain, which are optionally functionalized, comprising at least 4 carbon atoms and linked to the hydrocarbon-based units.

55. The process according to claim 54 wherein the keratin fibers are eyelashes.

56. A makeup kit comprising:
a first package comprising a first composition comprising at least 30% by weight water and/or a water-soluble solvent, and
a second package comprising a second composition comprising
a non-aqueous solvent phase,
at least one first polymer comprising
a) a polymer skeleton, having hydrocarbon-based repeating units comprising at least one hetero atom, and
optionally b) at least one pendent fatty chain and/or at least one terminal fatty chain, which are optionally functionalized, comprising at least 4 carbon atoms and linked to the hydrocarbon-based units.

57. A process for coating keratin fibers, comprising applying to the keratin fibers:
i) at least one first coat of a first composition with a dry extract of less than or equal to 50%,
ii) and then, after partial or total drying of the at least one first coat, at least one second coat of a second composition comprising
a non-aqueous solvent phase, and
at least one first polymer comprising
a) a polymer skeleton, having hydrocarbon-based repeating units comprising at least one hetero atom, and
optionally b) at least one pendent fatty chain and/or at least one terminal fatty chain, which are optionally functionalized, comprising at least 4 carbon atoms and linked to the hydrocarbon-based units.

58. The process according to claim 57, wherein the keratin fibers are eyelashes.

59. A makeup kit comprising:
a first package comprising a first composition with a dry extract of less than or equal to 50%,
a second package comprising a second composition comprising
a non-aqueous solvent phase, and
at least one first polymer comprising
a) a polymer skeleton, having hydrocarbon-based repeating units comprising at least one hetero atom, and
optionally b) at least one pendent fatty chain and/or at least one terminal fatty chain, which are optionally functionalized, comprising at least 4 carbon atoms and linked to the hydrocarbon-based units.
